(19) United States
(12) Patent Application Publication
(10) Pub. No.: US 2026/0193712 A1
(43) Pub. Date: Jul. 9, 2026

(54) Peripheral Blood Epigenetic Machine-Learning Models for Detecting Osteoarthritis of the Knee

(71) Applicant: Oklahoma Medical Research Foundation, Oklahoma City, OK (US)

(72) Inventor: Matlock Jeffries, Oklahoma City, OK (US)

(21) Appl. No.: 19/429,401

(22) Filed: Dec. 22, 2025

Related U.S. Application Data
(60) Provisional application No. 63/742,789, filed on Jan. 7, 2025.

(51) Int. Cl. C12Q 1/6883; G16B 20/20; G16B 40/20

(52) U.S. Cl. CPC C12Q 1/6883; G16B 20/20; G16B 40/20; C12Q 2600/118; C12Q 2600/154; C12Q 2600/156

(57) ABSTRACT

Provided herein are methods for determining whether a subject is at increased risk of developing future knee osteoarthritis, the method comprising the steps of: (a) obtaining, or having obtained, a blood sample; (b) measuring in the blood sample a DNA methylation level at one or more DNA methylation sites; (c) comparing the DNA methylation level(s) measured in step (b) to level(s) of DNA methylation in a control blood sample from an individual without osteoarthritis; (d) using a machine learning algorithm to generate an osteoarthritis risk score indicative of the likelihood of developing knee osteoarthritis based on the comparison of methylation levels; and (e) based on the osteoarthritis risk score identifying the subject as being at increased risk for future knee osteoarthritis and administering a treatment.

### CROSS-REFERENCE TO RELATED APPLICATIONS

This application claims priority to U.S. Provisional Application Ser. No. 63/742,789 filed Jan. 7, 2025, the entire contents of which are incorporated herein by reference.

### STATEMENT OF FEDERALLY FUNDED RESEARCH

This invention was made with government support under R01AR076440 awarded by the National Institutes of Health. The government has certain rights in the invention.

### TECHNICAL FIELD OF THE INVENTION

The present invention relates in general to the field of detecting osteoarthritis, and more particularly, to novel peripheral blood epigenetic machine-learning models that allow detection of osteoarthritis up to 96 months in advance.

### INCORPORATION-BY-REFERENCE OF MATERIALS FILED ON COMPACT DISC

The Sequence Listing in an XML file, named as _.xml of _KB, created on ______, and submitted to the United States Patent and Trademark Office via Patent Center, is incorporated herein by reference.

### BACKGROUND OF THE INVENTION

Without limiting the scope of the invention, its background is described in connection with osteoarthritis.

Osteoarthritis (OA) is a chronic, debilitating musculoskeletal disease characterized by progressive loss of function in both load-bearing and non-load-bearing joints leading to significant pain, mobility loss, and functional limitation, and is one of the most common diseases associated with aging. OA is the leading cause of chronic disability in the US (1) and is expected to affect more than 1 billion individuals worldwide by 2050 (2). The incidence of severe osteoarthritis requiring joint replacement is increasing at a rate that exceeds expected increases due to obesity and aging (3). Despite its prevalence, there are no disease-modifying anti-osteoarthritic drugs (DMOADs) approved by the US Food and Drug Administration (FDA) in stark contrast to the multitude of treatments available for other forms of arthritis.

A major impediment to therapeutic development and clinical trial design for knee OA, is an inability to diagnose early OA. Consensus definitions generally require radiographic changes, which tend to occur years into disease pathogenesis (4). Thus, disease-modifying therapies in trials are administered to patients who already have significant structural joint damage which may limit their ability to regenerate. Additionally, the lack of preclinical biomarkers has been a major obstacle in the implementation of OA prevention studies. This stands in contrast to studies of autoimmune forms of arthritis, e.g. rheumatoid arthritis, which have demonstrated significant benefit in delaying the onset of clinical arthritis in at-risk patients treated with early disease-modifying immunotherapy (5).

To address this shortcoming, several recent studies have sought to identify biomarkers of incident knee OA. Using 100 incident OA and 100 control subjects, Kraus and colleagues recently published a study of serum biomarkers to predict incident knee OA 8 years in advance (6), which demonstrated a reasonable receiver operator characteristic-area under the curve (ROC-AUC) of 0.76 using biomarker data alone. This increased to an AUC=0.83 when demographics, pain measures and hip OA indices were included. A similar study evaluating serum biomarkers published by Paz-Gonzalez and colleagues, yielded an AUC=0.83 (7), albeit in a smaller cohort (29 incident OA with 253 controls). Other groups have developed radiographic image-based models with AUCs ranging from 0.64 to 0.79 when demographic and other clinical variables were included (8,9).

A major drawback of these biochemical and radiographic biomarker approaches is their inherent variability from a standpoint of data collection and analysis. Despite these advances, a need remains for the early detection, prevention and treatment of osteoarthritis using novel biomarkers.

### SUMMARY OF THE INVENTION

As embodied and broadly described herein, an aspect of the present disclosure relates to a method for determining whether a subject is at increased risk of developing future knee osteoarthritis within 12 to 96 months, the method comprising the steps of: (a) obtaining, or having obtained, a blood sample; (b) measuring in the blood sample a DNA methylation level at one or more DNA methylation sites; (c) comparing the DNA methylation level(s) measured in step (b) to level(s) of DNA methylation in a control blood sample from an individual without osteoarthritis; (d) using a machine learning algorithm to generate an osteoarthritis risk score indicative of a likelihood of developing knee osteoarthritis based on the comparison of methylation levels; and (e) based on the osteoarthritis risk score identifying the subject as being at increased risk for future knee osteoarthritis within 12 to 96 months. In one aspect, the blood sample comprises peripheral blood leukocytes. In another aspect, the method further comprises at least one of: preprocessing a methylation array data by: excluding from analysis CpG probes located on sex chromosomes, probes with known single nucleotide polymorphisms (SNPs) with minor allele frequency of ≥5%, and probes not detected in all samples; or wherein the individual without osteoarthritis: (i) is matched to the subject by at least one of: age category, sex, BMI category, ethnicity, or baseline KL radiographic grade; and (ii) does not have osteoarthritis as determined by both radiographic and symptomatic examination. In another aspect, the machine learning algorithm is selected from a generalized logistic model, a parsimonious model, or combinations thereof. In another aspect, a control blood level of the DNA methylation is a predetermined standard from an individual that will not develop osteoarthritis within the subsequent 96 months as determined by radiographic and symptomatic examination. In another aspect, the DNA methylation is determined with an Illumina 450k, an EPIC v1, an EPIC v2 array, or CpG sites associates with at least one of: cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; or cg07592337. In another aspect, the DNA methylation analyzed further comprises in order of importance 1, 2, 3, 4, 5, 6, 7, 8, 9, 10, 11, 12, 13, 14, 15, 16, 17, 18, 19, 20, 25, 30, or 33 of the following biomarkers cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; and cg07592337. In another aspect, the subject is human. In another aspect, the subject has a Kellgren-Lawrence (KL) score of 0-1 in at least one knee. In another aspect, the method urther comprises administering at least one of acetaminophen, steroids, hyaluronic acid, nonsteroidal anti-inflammatory drugs (NSAIDs), or duloxetine, once the subject has a KL score of ≥2. In another aspect, the DNA methylation is a CpG island associated with 1, 2, 3, 4, 5, 6, 7, 8, or 9 markers selected from L3MBTL Histone Methyl-Lysine Binding Protein 1 (L3MBTL1), Urate oxidase (UOX), SEPTIN9, Toll-Interacting Protein (TOLLIP), Glycosyltransferase 1 Domain-Containing Protein 1 (GLT1D1), Ankyrin 3 (ANK3), Regulator Of G Protein Signaling 9 Binding Protein (RGS9BP), Interferon Alpha Inducible Protein 27 Like 1 (IFI27L1), and Glycogen Phosphorylase, Muscle Form (PYGM). In another aspect, the method further comprises administering to the subject with the osteoarthritis risk score identified as being at increased risk for future knee osteoarthritis at least one of acetaminophen, steroids, hyaluronic acid, nonsteroidal anti-inflammatory drugs (NSAIDs), or duloxetine. In another aspect, the knee osteoarthritis risk score model is generated by obtaining a complete dataset of knee osteoarthritis samples and expression data, selecting features using lasso regression (L1 penalization of a generalized logistic regression model) and CpG sites were identified as highly predictive of incident OA cases vs. controls; applying a Monte Carlo cross-validation step in which the data are split into 60 to 80% development and 20 to 40% validation subsets and modeling performed on the development set with a similar 10-fold cross-validated approach; splitting randomly 1:10 the data in the 60 to 80% development step datal applying a 10-fold cross-validated model development of the CpG+/− covariates available from glmnet models, repeating the random split 5, 6, 7, 8, 9, 10, 11, 12, 13, 14, or 15 times; after the nth repeat conducting further feature reduction and model tuning; optimizing the model 28, by testing against unseen data as a validation step. In another aspect, the method comprises cross-validating by looping random data splitting and model development for a total of 10, 20, 25, 30, 35, 40, 45, 50, or 60 cycles by Monte Carlo cross-validation, and splitting the data again split into random groups and is split into the Monte Carlo cross-validation step, and calculating a mean model performance for CpG sites selected to obtain the CpG sites used to calculate the knee osteoarthritis risk score. In another aspect, the method further comprises applying the knee osteoarthritis risk score model to a lockbox set to determine at least one of area under the receiver operator characteristic curve (AUC), diagnostic odds ratio, accuracy, sensitivity, specificity, or F1 score; and DNA methylation sites.

As embodied and broadly described herein, an aspect of the present disclosure relates to a method for determining whether to treat a subject at increased risk of developing future knee osteoarthritis within 12 to 96 months, the method comprising the steps of: (a) obtaining, or having obtained, DNA methylation level(s) at one or more CpG sites from a blood sample of the subject; (b) comparing the DNA methylation level(s) measured in step (a) to DNA methylation level(s) at one or more CpG sites in a control blood sample from an individual without osteoarthritis; (c) calculating using a machine learning algorithm to generate an osteoarthritis risk score indicative of risk of future knee osteoarthritis based on the comparison of methylation levels; (d) based on the osteoarthritis risk score identifying the subject as being at increased risk for future knee osteoarthritis within 12 to 96 months with the osteoarthritis risk score generated in step (c) that is indicative that the subject is at risk of knee osteoarthritis within 12 to 96 months; and (e) if the subject has osteoarthritis risk score for increased risk and a Kellgren-Lawrence (KL) score of ≥2, then administering to the subject with at least one of acetaminophen, steroids, hyaluronic acid, nonsteroidal anti-inflammatory drugs (NSAIDs), or duloxetine in an amount effective to reduce knee osteoarthritis. In one aspect, the subject is at increased risk of knee osteoarthritis and has a Kellgren-Lawrence (KL) score of 0-1 in at least one knee. In another aspect, the blood sample obtained from the subject has been prepared in the presence of heparin. In another aspect, the machine learning algorithm is selected from a generalized logistic model, a parsimonious model, or combinations thereof. In another aspect, a control blood level of the DNA methylation is a predetermined standard from an individual that does not have osteoarthritis as determined by both radiographic and symptomatic examination. In another aspect, the DNA methylation is determined with an Illumina 450k, an EPIC v1, an EPIC v2 array, or CpG sites associates with at least one of: cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; or cg07592337. In another aspect, the DNA methylation analyzed further comprises in order of importance 1, 2, 3, 4, 5, 6, 7, 8, 9, 10, 11, 12, 13, 14, 15, 16, 17, 18, 19, 20, 25, 30, or 33 of the following biomarkers cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; and cg07592337. In another aspect, the DNA methylation level(s) in the control blood sample is a level of DNA methylation in a similarly processed blood sample from an individual without osteoarthritis or a mean level of several individuals without osteoarthritis. In another aspect, the individual without osteoarthritis (i) is matched to the subject by at least one of: age category, sex, BMI category, ethnicity, or baseline KL radiographic grade; and (ii) does not have osteoarthritis as determined by both radiographic and symptomatic examination. In another aspect, the DNA methylation is a CpG island associated with 1, 2, 3, 4, 5, 6, 7, 8, or 9 markers selected from L3MBTL Histone Methyl-Lysine Binding Protein 1 (L3MBTL1), Urate oxidase (UOX), SEPTIN9, Toll-Interacting Protein (TOLLIP), Glycosyltransferase 1 Domain-Containing Protein 1 (GLT1D1), Ankyrin 3 (ANK3), Regulator Of G Protein Signaling 9 Binding Protein (RGS9BP), Interferon Alpha Inducible Protein 27 Like 1 (IFI27L1), and Glycogen Phosphorylase, Muscle Form (PYGM). In another aspect, further comprising administering to the subject with the osteoarthritis risk score identified as being at increased risk for future knee osteoarthritis at least one of acetaminophen, steroids, hyaluronic acid, nonsteroidal anti-inflammatory drugs (NSAIDs), or duloxetine. In another aspect, the knee osteoarthritis risk score model is generated by obtaining a complete dataset of knee osteoarthritis samples and expression data, selecting features using lasso regression (L1 penalization of a generalized logistic regression model) and CpG sites were identified as highly predictive of incident OA cases vs. controls; applying a Monte Carlo cross-validation step in which the data are split into 60 to 80% development and 20 to 40% validation subsets and modeling performed on the development set with a similar 10-fold cross-validated approach; splitting randomly 1:10 the data in the 60 to 80% development step datal applying a 10-fold cross-validated model development of the CpG+/− covariates available from glmnet models, repeating the random split 5, 6, 7, 8, 9, 10, 11, 12, 13, 14, or 15 times; after the nth repeat conducting further feature reduction and model tuning; optimizing the model 28, by testing against unseen data as a validation step. In another aspect, the method comprises cross-validating by looping random data splitting and model development for a total of 10, 20, 25, 30, 35, 40, 45, 50, or 60 cycles by Monte Carlo cross-validation, and splitting the data again split into random groups and is split into the Monte Carlo cross-validation step, and calculating a mean model performance for CpG sites selected to obtain the CpG sites used to calculate the knee osteoarthritis risk score. In another aspect, the method further comprises applying the knee osteoarthritis risk score model to a lockbox set to determine at least one of: area under the receiver operator characteristic curve (AUC), diagnostic odds ratio, accuracy, sensitivity, specificity, or F1 score; and DNA methylation sites.

As embodied and broadly described herein, an aspect of the present disclosure relates to a method for determining whether a subject is at increased risk of developing future knee osteoarthritis, wherein the subject does not have osteoarthritis but is predicted to have knee osteoarthritis in 0 to 96 months, the method comprising the steps of: (a) comparing DNA methylation level(s) measured at one or more CpG sites from a blood sample of the subject to DNA methylation level(s) at one or more CpG sites in a control blood sample from an individual without osteoarthritis; (b) calculating using a machine learning algorithm to generate an osteoarthritis risk score, wherein the machine learning algorithm is selected from a generalized logistic model, a parsimonious model, or combinations thereof; and (c) based on the osteoarthritis risk score identifying the subject as being at increased risk for developing future osteoarthritis of the knee within 0 to 96 months when the osteoarthritis risk score is equal to, or greater than, 73% accuracy, wherein the DNA methylation is determined for at least 1, 2, 3, 4, 5, 6, 7, 8, 9, or 10 markers selected from: cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; or cg07592337. In one aspect, the DNA methylation analyzed further comprises in order of importance 1, 2, 3, 4, 5, 6, 7, 8, 9, 10, 11, 12, 13, 14, 15, 16, 17, 18, 19, 20, 25, 30, or 33 of the following biomarkers cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; and cg07592337. In another aspect, the methylation of the 1, 2, 3, 4, 5, 6, 7, 8, 9, or 10 markers is selected, in the following order: cg06908806; cg13731777; cg17976021; cg01095449; cg14471784; cg06601130; cg19922101; cg14938831; cg27387124; and cg05102645. In another aspect, the subject has not been diagnosed with knee osteoarthritis and/or has a Kellgren-Lawrence (KL) score of 0-1. In another aspect, further comprising administering to the subject with the osteoarthritis risk score identified as being at increased risk for future knee osteoarthritis at least one of acetaminophen, steroids, hyaluronic acid, nonsteroidal anti-inflammatory drugs (NSAIDs), or duloxetine. In another aspect, the knee osteoarthritis risk score model is generated by obtaining a complete dataset of knee osteoarthritis samples and expression data, selecting features using lasso regression (L1 penalization of a generalized logistic regression model) and CpG sites were identified as highly predictive of incident OA cases vs. controls; applying a Monte Carlo cross-validation step in which the data are split into 60 to 80% development and 20 to 40% validation subsets and modeling performed on the development set with a similar 10-fold cross-validated approach; splitting randomly 1:10 the data in the 60 to 80% development step datal applying a 10-fold cross-validated model development of the CpG+/− covariates available from glmnet models, repeating the random split 5, 6, 7, 8, 9, 10, 11, 12, 13, 14, or 15 times; after the nth repeat conducting further feature reduction and model tuning; optimizing the model 28, by testing against unseen data as a validation step. In another aspect, the method comprises cross-validating by looping random data splitting and model development for a total of 10, 20, 25, 30, 35, 40, 45, 50, or 60 cycles by Monte Carlo cross-validation, and splitting the data again split into random groups and is split into the Monte Carlo cross-validation step, and calculating a mean model performance for CpG sites selected to obtain the CpG sites used to calculate the knee osteoarthritis risk score. In another aspect, the method further comprises applying the knee osteoarthritis risk score model to a lockbox set to determine at least one of: area under the receiver operator characteristic curve (AUC), diagnostic odds ratio, accuracy, sensitivity, specificity, or F1 score; and DNA methylation sites.

As embodied and broadly described herein, an aspect of the present disclosure relates to a computer-implemented system for determining whether a subject is at increased risk of developing future knee osteoarthritis within 12 to 96 months comprising: a hardware processor and a memory coupled to the hardware processor, wherein the memory comprises a set of instructions in the form of a processing subsystem, configured to be executed by the hardware processor, comprises: obtaining, or having obtained, a blood sample; measuring in the blood sample a DNA methylation level at one or more DNA methylation sites; comparing the DNA methylation level(s) measured in step (b) to level(s) of DNA methylation in a control blood sample from an individual without osteoarthritis; using a machine learning algorithm to generate an osteoarthritis risk score indicative of a likelihood of developing knee osteoarthritis based on the comparison of methylation levels; and based on the osteoarthritis risk score identifying the subject as being at increased risk for future knee osteoarthritis within 12 to 96 months. In one aspect, the computer-implemented system further comprises the step of administering to the subject with osteoarthritis risk score indicative of a likelihood of developing knee osteoarthritis at least one of acetaminophen, steroids, hyaluronic acid, nonsteroidal anti-inflammatory drugs (NSAIDs), or duloxetine, once the subject has a KL score of ≥2. In another aspect, the knee osteoarthritis risk score model is generated by obtaining a complete dataset of knee osteoarthritis samples and expression data, selecting features using lasso regression (L1 penalization of a generalized logistic regression model) and CpG sites were identified as highly predictive of incident OA cases vs. controls; applying a Monte Carlo cross-validation step in which the data are split into 60 to 80% development and 20 to 40% validation subsets and modeling performed on the development set with a similar 10-fold cross-validated approach; splitting randomly 1:10 the data in the 60 to 80% development step datal applying a 10-fold cross-validated model development of the CpG+/− covariates available from glmnet models, repeating the random split 5, 6, 7, 8, 9, 10, 11, 12, 13, 14, or 15 times; after the nth repeat conducting further feature reduction and model tuning; optimizing the model 28, by testing against unseen data as a validation step. In another aspect, the method comprises cross-validating by looping random data splitting and model development for a total of 10, 20, 25, 30, 35, 40, 45, 50, or 60 cycles by Monte Carlo cross-validation, and splitting the data again split into random groups and is split into the Monte Carlo cross-validation step, and calculating a mean model performance for CpG sites selected to obtain the CpG sites used to calculate the knee osteoarthritis risk score. In another aspect, the method further comprises applying the knee osteoarthritis risk score model to a lockbox set to determine at least one of: area under the receiver operator characteristic curve (AUC), diagnostic odds ratio, accuracy, sensitivity, specificity, or F1 score; and DNA methylation sites.

As embodied and broadly described herein, an aspect of the present disclosure relates to a non-transitory computer-readable medium storing a computer program that, when executed by a processor, causes the processor to perform a method for determining whether a subject is at increased risk of developing future knee osteoarthritis within 12 to 96 months, wherein the method comprises: interfacing with a machine learning model that generate a knee osteoarthritis risk score; automatically processing, a knee osteoarthritis risk score module by: obtaining, or having obtained, a blood sample; measuring in the blood sample a DNA methylation level at one or more DNA methylation sites; comparing the DNA methylation level(s) measured in step (b) to level(s) of DNA methylation in a control blood sample from an individual without osteoarthritis; using a machine learning algorithm to generate an osteoarthritis risk score indicative of a likelihood of developing knee osteoarthritis based on the comparison of methylation levels; and based on the osteoarthritis risk score identifying the subject as being at increased risk for future knee osteoarthritis within 12 to 96 months. In another aspect, the knee osteoarthritis risk score model is generated by obtaining a complete dataset of knee osteoarthritis samples and expression data, selecting features using lasso regression (L1 penalization of a generalized logistic regression model) and CpG sites were identified as highly predictive of incident OA cases vs. controls; applying a Monte Carlo cross-validation step in which the data are split into 60 to 80% development and 20 to 40% validation subsets and modeling performed on the development set with a similar 10-fold cross-validated approach; splitting randomly 1:10 the data in the 60 to 80% development step datal applying a 10-fold cross-validated model development of the CpG+/− covariates available from glmnet models, repeating the random split 5, 6, 7, 8, 9, 10, 11, 12, 13, 14, or 15 times; after the nth repeat conducting further feature reduction and model tuning; optimizing the model 28, by testing against unseen data as a validation step. In another aspect, the method comprises cross-validating by looping random data splitting and model development for a total of 10, 20, 25, 30, 35, 40, 45, 50, or 60 cycles by Monte Carlo cross-validation, and splitting the data again split into random groups and is split into the Monte Carlo cross-validation step, and calculating a mean model performance for CpG sites selected to obtain the CpG sites used to calculate the knee osteoarthritis risk score. In another aspect, the method further comprises applying the knee osteoarthritis risk score model to a lockbox set to determine at least one of: area under the receiver operator characteristic curve (AUC), diagnostic odds ratio, accuracy, sensitivity, specificity, or F1 score; and DNA methylation sites.

### BRIEF DESCRIPTION OF THE DRAWINGS

For a more complete understanding of the features and advantages of the present invention, reference is now made to the detailed description of the invention along with the accompanying figures and in which:

FIG. 1 is a diagram of machine learning model development workflow of the present disclosure.

FIGS. 2A to 2D show the generalized logistic model performance under various conditions to classify samples as incident OA or healthy control. FIG. 2A: Using demographic data only (age, sex, BMI, ethnicity, smoking history, NSAID use history, narcotic use history). FIG. 2B: Using imputed peripheral blood cellular composition data only. FIG. 2C: Performance of DNA methylation-based models using development data. FIG. 2D: Performance of DNA methylation-based models using validation data not used during development.

FIGS. 3A to 3D show the performance of generalized logistic models in secondary outcomes. FIG. 3A: Using 13 CpGs identified in previous study predicting future progression in established OA patients to predict incident status. FIG. 3B: Using CpGs associated with genes encoding proteins identified as predictive of incident status in other studies. FIG. 3C: Using CpGs from the current incident work to predict future progression in established OA patients. FIG. 3D: Using genotype data (SNPs) from the GeCKO genotyping study in these same patients.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

To overcome the problems in the prior art, the inventors determined whether peripheral blood DNA methylation modeling might also be applicable to the study of incident knee OA, leveraging data and biospecimens from the Osteoarthritis Initiative (OAI). It was found that incident radiographic osteoarthritis can be predicted by peripheral blood epigenetic machine-learning models up to 96 months in advance.

Study design, incident radiographic knee OA definition, DNA methylation quantitation. Samples and data were obtained from the Osteoarthritis Initiative (OAI) study, where participants provided written informed consent. The present study was then approved by the Institutional Review Board of the Oklahoma Medical Research Foundation. Participants had baseline and yearly follow-up knee radiographs and baseline buffy coat DNA available. Kellgren-Lawrence Grade (KLG)(18) was assessed by a central reading site using non-fluoroscopic fixed-flexion knee radiographs with a Synaflexer positioning device (Synarc, Newark, CA). All buffy coat DNA samples were derived from the baseline visit blood draw. The OAI dataset was queried for individuals who at baseline had Kellegren-Lawrence grade (KLG) knee radiograph scores of 0-1 or KLG=2 without joint space narrowing (JSN) but then developed incident KLG≥2 with JSN of either knee within 24-96 months. Controls were age±5 years, BMI±5 units, sex, and (with two exceptions) ethnicity-matched and consisted of patients with KLG 0-1 with or without JSN or KLG=2 without JSN at all follow-up timepoints through 96 months. Demographic variables (including age, sex, Caucasian, African American, Asian American and Hispanic ancestry), body mass index (BMI), baseline Western Ontario and McMasters pain index (WOMAC pain), non-steroidal anti-inflammatory drug use and opiate pain medication use, were obtained and compared among progressor groups.

DNA samples used in this study were derived from buffy coat and were previously extracted by OAI personnel, stored within the OAI biobank, and shipped to the inventors for further analysis. For methylation analysis, 500 ng of DNA was treated with sodium bisulfite (EZ DNA methylation kit, Zymo) and loaded onto Illumina Infinium EPICv2 methylation arrays. Arrays were imaged by the Clinical Genomics Center at OMRF in 6 batches; each batch consisted of an equal number of incident cases and controls, and each methylation array chip had an even distribution of cases and controls.

Data preprocessing. Statistical analysis was performed using R (v. 4.4.0). Raw IDAT files were loaded and processed using the minfi package (v.1.50.0). Raw array data were first loaded, normalization performed using the functional normalization method, and CpG site methylation data converted to beta values (0-1 methylation value estimate representing the ratio of methylated to unmethylated probe intensities at a given CpG site); probes with detection P≥0.01 were dropped. From an initial set of 930,075 probes, the following were excluded: probes targeting non-CpG positions, probes located on sex chromosomes, and probes with known single nucleotide polymorphisms within 5 bp of the 3′ end of the CpG probe with any minor allele frequency (MAF>0%), and probes not detected in every sample (N=41,594). Some probes on EPICv2 arrays are duplicated (N=6,048); thus, these were collapsed to a single measure (keeping the mean methylation value of duplicated probes), leaving 882,433 CpG probes for analysis. Sex was predicted based on methylation data using the getSex( ) function of minfi. The initial dataset consisted of 472 samples (236 cases and 236 controls); however, one case and the associated matched control were dropped due to low quality methylation data, and one additional case and control were dropped due to a mismatch in predicted and actual sex, leaving 234 cases and 234 controls for subsequent analysis. Of note, the inventors also performed batch effects analysis and correction using the BEclear package (19), but this did not change modeling results compared to non-batch-corrected data (P=1.0); thus, non-batch-corrected data were used for analysis.

Cellular composition estimation. Currently available cellular composition deconvolution models using DNA methylation microarray data are limited to inputs of historic Illumina array generations (27k, 450k, EPICv1), rather than the most recent microarray version used in the present study (EPICv2). Thus, the inventors developed new methylation-based decomposition models that could be applied to EPICv2 data. First, cellular composition of 12 cell subsets was estimated using previously published libraries (20) on the inventors' previous dataset of 694 established OA patients (17). Then, the established OA dataset was subset to only those CpGs shared with the newest EPICv2 dataset generated in the current study (N=700,248 CpGs), and 10-fold cross-validated generalized Gaussian logistic models were developed using cv.glmnet for each cellular subset independently; a total of 2,977 CpG sites were used by at least one cellular prediction model. These models performed similarly to the reference imputation method (R²=0.9995). These models were then applied to the EPICv2 dataset generated in the current study and group differences determined. As data were non-normally distributed, group differences were calculated using a Mann-Whitney test with Benjamin-Hochberg post-hoc FDR correction.

Model development. For model development, the inventors created significant improvements to their previously published approach (17). The prior modeling approach was not optimized for selection of individual methylation sites (i.e., during the initial ‘screen’ of all methylation sites). In the present disclosure, the inventors updated the modeling approach as well as modeling a different outcome, namely, future OA development in healthy individuals as opposed to predicting progression in individuals already diagnosed with OA.

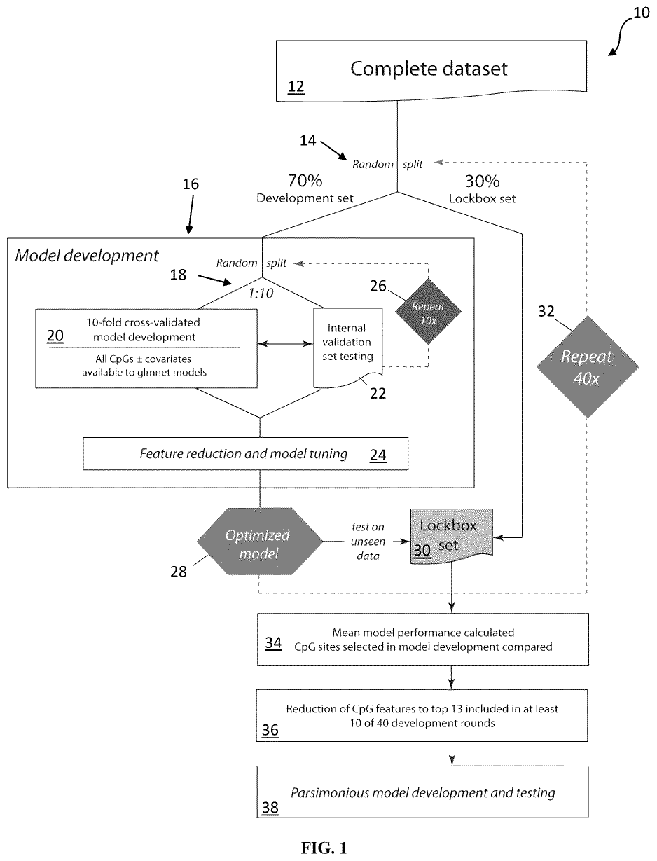

FIG. 1 is a diagram of machine learning model to generate a knee osteoarthritis risk score 10 development workflow of the present disclosure. First, in step 12, a complete dataset is obtained, as described in detail hereinbelow (Table 1). Feature selection was implemented using lasso regression (L1 penalization of a generalized logistic regression model) and 33 CpG sites were identified as highly predictive of incident OA cases vs. controls. Next, in step 14, the data from step 12 were randomly split into a 70% development and a 30% lockbox subsets and modeling performed on the development set (step 16) using a similar 10-fold cross-validated approach. At step 18, the 70% development step data is split 1:10 and the data that is randomly split, at step 20 enters a 10-fold cross-validated model development and the 33 CpGs+/− covariates available from glmnet models, while an internal validation set testing is conducted at step 22. In step 26, the random split is repeated 10 times. After the 10^{th} repeat, in step 24, the data enter the feature reduction and model tuning. The optimized model 28, is then tested in unseen data at step 30, which is the lockbox set. Further cross-validation was performed by looping random data splitting and model development for a total of 40 cycles (Monte Carlo cross-validation). At step 32, the data is again split into random groups and is split into the Monte Carlo cross-validation step, the data from step 14 were split into 70% development and 30% validation subsets and modeling performed again. From the lockbox set 30, the data is then processed as follows: At step 34, the mean model performance is calculated and CpG sites selected in the model development are compared. At step 36, a reduction of CpG features to the top 13 is included in at least 10 f the 40 development rounds. Finally, at step 38, the parsimonious model development and testing is complete. Models were then applied to the lockbox set, and performance characteristics including area under the receiver operator characteristic curve (AUC), diagnostic odds ratio, accuracy, sensitivity, specificity, or F1 score (the weighted average of precision and recall) were recorded. DNA methylation sites (features) selected for inclusion in each model were recorded and compared.

Thus, the knee osteoarthritis risk score model is generated by obtaining a complete dataset of knee osteoarthritis samples and expression data, selecting features using lasso regression (L1 penalization of a generalized logistic regression model) and CpG sites were identified as highly predictive of incident OA cases vs. controls; applying a Monte Carlo cross-validation step in which the data are split into 60, 65, 70, 75, or 80% development and 20, 25, 30, 35, 40% validation subsets and modeling performed on the development set with a similar 10-fold cross-validated approach; splitting randomly 1:10 the data in the 60, 65, 70, 75, or 80% (e.g., 70%) development step datal applying a 10-fold cross-validated model development of the CpG+/− covariates available from glmnet models, repeating the random split 5, 6, 7, 8, 9, 10, 11, 12, 13, 14, or 15 times; after the nth repeat conducting further feature reduction and model tuning; optimizing the model 28, by testing against unseen data as a validation step. In another aspect, the method comprises cross-validating by looping random data splitting and model development for a total of 10, 20, 25, 30, 35, 40, 45, 50, or 60 cycles by Monte Carlo cross-validation, and splitting the data again split into random groups and is split into the Monte Carlo cross-validation step, and calculating a mean model performance for CpG sites selected to obtain the CpG sites used to calculate the knee osteoarthritis risk score. The method further comprises applying the knee osteoarthritis risk score model to a lockbox set to determine at least one of: area under the receiver operator characteristic curve (AUC), diagnostic odds ratio, accuracy, sensitivity, specificity, or F1 score; and DNA methylation sites.

Baseline demographic, disease, and peripheral blood cellular composition characteristics were well matched; models developed using only patient characteristics or imputed cellular composition performed poorly.

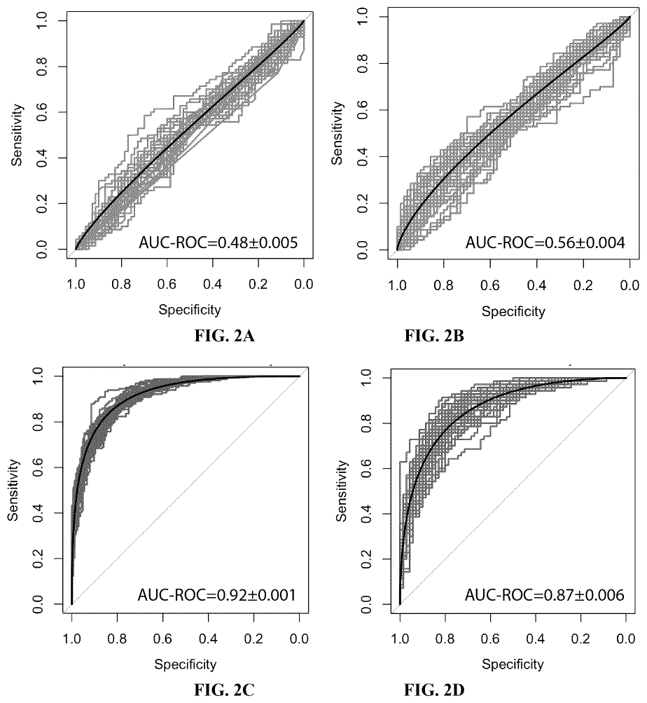

Baseline patient characteristics were well matched between incident cases and controls (Table 1). Models developed on patient characteristic data alone did not differentiate cases from controls (ROC-AUC 0.48±0.005, mean±SEM, model accuracy 0.48±0.005, FIG. 2A). Mixed peripheral blood DNA methylation analyses can be confounded by changes in cellular composition among study groups, as immune cell subsets have distinct epigenetic signatures (21). Cell composition estimation can be performed using DNA methylation microarray data; e.g. as implemented in the estimateCellCounts function of minfi R package (22); however, these composition functions have not yet been updated to support the EPICv2 arrays. Thus, to analyze imputed cellular composition in the present study, the inventors developed new models for the 12 peripheral blood immune cell subsets defined in a previous publication (20). First, the inventors defined immune cell subset composition using previous methods in data from 554 OA patients from the OAI in which the inventors have previously published EPICv1 array data (17); then, the inventors trained 10-fold cross-validated generalized logistic regression models for each cellular subset on a reduced methylation dataset containing CpGs present in both these previous 554 samples and the 468 EPICv2 samples from the present study (n=702,924 CpGs available to models, parsimonious cell composition model set n=2,766 CpGs used by at least one cell type model). It was confirmed that the novel models exhibited similar performance to the reference models (R²=0.9995, mean error of imputed model vs. reference models 0.22±0.002%, mean±SEM). Finally, the inventors applied these new models to the samples in the present study and compared composition among the two groups. The inventors did not find any statistically significant differences in cell counts in incident OA cases vs. controls, although two cell types nearly met statistical significance (eosinophils in incident cases: 1.7±0.1% vs. healthy controls 1.4±0.1, q=0.08, monocytes in incident cases 9.0±0.2% vs. healthy controls 8.5±0.1, q=0.08, Table 2). To further analyze whether these variations could be useful in predicting incident status, a modeling approach was used on the methylation data using cell composition data but did not find a robust discriminatory capability: (ROC-AUC 0.56±0.004, mean±SEM, accuracy 0.52±0.004, FIG. 2B).

TABLE 1
Participant characteristics and imputed
peripheral blood cell composition

| | Incident | | Healthy | | |
|---|---|---|---|---|---|
| | radiographic | | controls | | |
| | OA (n = 234), | | (n = 234), | | |
| | mean ± SEM | | mean ± SEM | | |
| | (%) | | (%) | | P values |
| Age (mean ± SD), | 60.6 ± 8.5 | | 60.6 ± 8.6 | | 0.98 |
| years | | | | | |
| Sex (% female) | 68% | | 68% | | 1.00 |
| BMI (mean ± SD), | 29.2 ± 0.3 | | 28.8 ± 0.3 | | 0.27 |
| kg/m² | | | | | |
| White | 201 | (86%) | 201 | (86%) | 1.00 |
| African-American | 29 | (12%) | 31 | (13%) | 0.89 |
| Asian-American | 1 | (0.4%) | 1 | (0.4%) | 1.00 |
| Hispanic | 1 | (0.4%) | 3 | (1%) | 0.62 |
| Other non-white | 2 | (1%) | 0 | | 0.50 |
| Smoking history | 103 | (44%) | 115 | (49%) | 0.31 |
| (%) | | | | | |
| NSAID use (%) | 48 | (21%) | 41 | (18%) | 0.48 |
| Opiate use (%) | 5 | (2.1%) | 5 | (2.1%) | 1.00 |

TABLE 2
Imputed peripheral blood cell composition
Imputed peripheral blood cell composition

| | Incident | Healthy | | |
|---|---|---|---|---|
| | cases | controls | | |
| | (n = 234) | (n = 234) | | |
| | (mean ± | (mean ± | | |
| | SEM % | SEM % | Mann- | |
| | total | total | Whitney P | B&H q |
| | population) | population) | values | values |
| Basophil | 1.2 ± 0.1 | 1.0 ± 0.1 | 0.042 | 0.12 |
| Memory B cell | 2.5 ± 0.1 | 2.3 ± 0.1 | 0.098 | 0.24 |
| Naïve B cell | 4.2 ± 0.2 | 3.9 ± 0.1 | 0.35 | 0.69 |
| Memory CD4+ T | 14 ± 0.3 | 14 ± 0.3 | 0.68 | 0.77 |
| cell | | | | |
| Naïve CD4+ T | 5.9 ± 0.3 | 6.8 ± 0.3 | 0.030 | 0.12 |
| cell | | | | |
| Memory CD8+ T | 6.6 ± 0.4 | 6.1 ± 0.4 | 0.40 | 0.69 |
| cell | | | | |
| Naïve CD8+ T | 1.2 ± 0.1 | 1.3 ± 0.1 | 0.68 | 0.77 |
| cell | | | | |
| Eosinophil | 1.7 ± 0.1 | 1.4 ± 0.1 | 0.0073 | 0.081 |
| Monocyte | 9.0 ± 0.2 | 8.5 ± 0.1 | 0.014 | 0.081 |
| Neutrophil | 47 ± 0.7 | 48 ± 0.7 | 0.57 | 0.77 |
| Natural killer | 5.1 ± 0.1 | 5.1 ± 0.1 | 0.88 | 0.88 |
| (NK) T cell | | | | |
| Regulatory T cell | 1.3 ± 0.1 | 1.3 ± 0.1 | 0.71 | 0.77 |
| (Treg) | | | | |

Models developed using baseline peripheral blood DNA methylation data robustly

Next, the inventors investigated DNA methylation. First, the inventors determined which CpG sites were broadly predictive of incident OA using the feature reduction capabilities of glmnet by training a single 10-fold internally cross-validated generalized logistic model (glm), which identified 33 CpG sites (FIG. 1, Table 3). The inventors then reduced the dataset to focus on these 33 sites, split the data into 7000 development and 30% validation subsets, generated a 10-fold cross-validated glm model, and applied this model to both the development and validation datasets. The inventors then repeated this random 70/30 data split and model development for a total of 40 cycles. These models demonstrated robust predictive capability (development models: RGC-AUC 0.92±0.001, accuracy 0.83±0.002; validation models: RGC-AUC 0.87±0.004, accuracy 0.79±0.006), sensitivity 0.79±0.007, specificity 0.79±0.006, F1 score 0.79±0.006 (FIGS. 2C, 2D). Model accuracy was similar across the various times-to-incident-GA-development (24 m: accuracy 0.71, 48 m: 0.79, 48 m: 0.85, 72 m: 0.77, 96 m: 0.92, control: 0.84).

TABLE 3
CpG sites predictive of incident OA. CpG location listed relative to transcription
start site (listed in base pairs distant from TSS), CpG island location listed
relative to nearest CpG island, if applicable (N locations upstream of CpG island,
S locations downstream of CpG island). Relative contribution calculated as normalized
mean cv.glm model coefficient over 40 development cycles).

| | | | | | Mean relative |
|---|---|---|---|---|---|
| | | Gene | CpG | CpG | contribution to |
| CpG site | Chromosome | symbol | location | Island | incident models |
| cg06908806 | chr20 | L3MBTL1 | TSS150 | Island | 0.15 |
| cg13731777 | chr10 | | | | 0.13 |
| cg17976021 | chr1 | UOX | TSS1500 | | 0.08 |
| cg01095449 | chr3 | | | | 0.06 |
| cg14471784 | chr9 | | | | 0.05 |
| cg06601130 | chr2 | | | S_Shelf | 0.05 |
| cg19922101 | chr17 | SEPTIN9 | TSS1500 | S_Shore | 0.05 |
| cg14938831 | chr10 | | | | 0.04 |
| cg27387124 | chr5 | | | | 0.03 |
| cg05102645 | chr3 | | | | 0.03 |
| cg22424444 | chr11 | TOLLIP | Exon 1 | S_Shore | 0.03 |
| cg08111925 | chr15 | | | | 0.03 |
| cg12257696 | chr8 | | | | 0.03 |
| cg16478864 | chr12 | GLTID1 | Exon 8 | S_Shelf | 0.02 |
| cg07328664 | chr2 | | | | 0.02 |
| cg26395513 | chr7 | | | | 0.02 |
| cg02918525 | chr2 | | | | 0.02 |
| cg18016034 | chr10 | ANK3 | TSS200 | | 0.02 |
| cg12597363 | chr8 | | | N_Shore | 0.02 |
| cg15480200 | chr10 | | | | 0.02 |
| cg05946856 | chr6 | | | | 0.01 |
| cg27413643 | chr19 | RGS9BP | TSS1500 | Island | 0.01 |
| cg06736189 | chr18 | | | | 0.01 |
| cg27560391 | chr14 | IFI27L1 | TSS1500 | N_Shore | 0.01 |
| cg19093112 | chr9 | | | S_Shelf | 0.01 |
| cg21743307 | chr3 | | | S_Shelf | 0.006 |
| cg07553358 | chr3 | | | S_Shelf | 0.006 |
| cg13532410 | chr3 | | | N_Shelf | 0.005 |
| cg13606440 | chr3 | | | | 0.003 |
| cg05139637 | chr2 | | | | 0.002 |
| cg10972590 | chr8 | | | | 0.002 |
| cg22668906 | chr14 | | | | 0.001 |
| cg07592337 | chr11 | PYGM | | | 0.0003 |

Models based on previous OA progression prediction CpGs perform reasonably well at predicting future OA development, although methylation models based on previously-identified predictive serum proteins perform poorly. Thus, the machine learning model of the present disclosure improved significantly on prior model, plus, it further improved on the unreliable predictive serum proteins-based methods.

Next, the inventors sought to determine whether the same CpG sites identified in the inventors' previous work as predictive of future radiographic and/or pain progression in patients with established OA (n=13) (17) could also be used to predict incident OA.

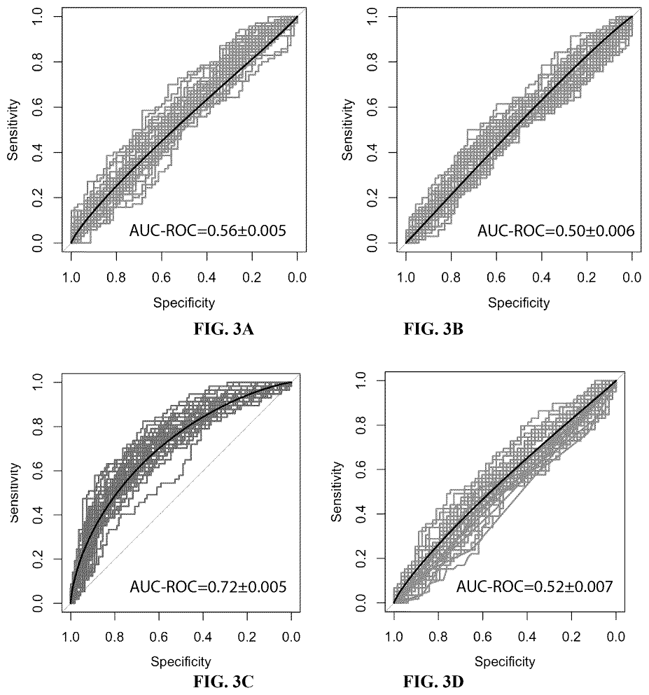

Thus, a subset of DNA methylation data was applied to the 13 CpGs identified as most predictive in a previous progression model, and developed the new models as described above. These models did not discriminate incident cases from controls (AUC-ROC=0.56±0.005, mean±SEM, accuracy 0.54±0.005, FIG. 3A). The inventors also sought to determine whether CpG sites associated with the 24 protein biomarkers recently identified as predictive of incident OA onset by Kraus et al. (6) might also be predictive. 148 CpGs were identified in the data that were associated with these genes, and developed new models as described above using these as input data. These models did not discriminate incident cases from controls (AUC-ROC=0.50±0.006, mean±SEM, accuracy 0.49±0.006, FIG. 3B), and were in fact significantly worse than the protein markers themselves as reported by Kraus (AUC-ROC=0.76).

CpGs that predict incident radiographic OA are also useful to predict future progression in established OA.

Next, the inventors evaluated the possibility that the 33 CpGs the inventors identified as predictive of incident OA might be useful in predicting future radiographic progression in established OA. Thus, the inventors went back to the inventors' prior peripheral blood methylation dataset in established OA patients (17) and focused on those patients who went on to experience rapid radiographic progression within the subsequent 48 months compared to non-progressive controls. Of the 33 CpGs, the inventors identified in the present study as predictive of incident OA, 23 were available in the previous-generation EPICv1 methylation arrays was used in the progression study; thus, a subset of the data of these 23 CpGs was used and rederived models were generated to predict future progression as described above. These models performed well (AUC-ROC 0.72±0.005, accuracy 0.70±0.004, sensitivity 0.71±0.003, specificity 0.66±0.01, F1 score 0.80±0.002, FIG. 3C).

Genotype is not predictive of future incident OA in the cohort. Finally, the inventors evaluated whether genetic data, either on their own or in addition to methylation data, might be useful in the current cohort to predict incident OA. To do this, the inventors accessed the Genetic Components of Knee Osteoarthritis (GeCKO) study data, which performed SNP genotyping using Illumina HumanOmni2.5-4v1_B arrays on most OAI patients. SNP data (n=366,510 SNPs) were available for 409 of the subjects. The inventors then performed modeling as above on SNP data alone, but these models performed poorly (AUC-ROC=0.52±0.007, mean±SEM, accuracy 0.52±0.006, FIG. 3D).

The inventors sought to evaluate whether peripheral blood-based epigenetic biomarkers in healthy individuals could be used to predict the development of future incident radiographic OA within 24-96 months. To do this, the inventors generated genome-wide DNA methylation data on baseline peripheral blood buffy coat cells using Illumina MethylationEPICv2 arrays in 468 patients from the OAI, including 234 incident cases and 234 matched controls. The inventors then applied machine learning techniques to both, identify individual methylation sites that were associated with incident OA, as well as develop models to differentiate incident cases from controls. To reduce overfitting, models were trained on a randomly selected 70% development dataset and tested on a 30% validation dataset, then results were averaged over 40 such cycles of development-validation data splits in a Monte Carlo cross-validation methodology.

To confirm that the case and control matching was adequate, the inventors first derived models based solely on clinical characteristics, which the inventors found were not predictive of incident OA status. The inventors also used genotyping array data from the Genetic Components of Knee Osteoarthritis (GeCKO) study, which covered most of the samples (n=408 of 468 total samples) and modeled outcomes on these data, but these models were also not predictive of incident OA. A major concern when performing DNA methylation analysis on a mixed sample, particularly peripheral blood, is that methylation changes can be seen due to differences in cellular composition rather than de novo methylation differences between groups. Although peripheral blood composition data were not directly determined in the OAI, there have been previous methods published that estimate cellular composition based on genome-wide methylation arrays; the most recent and extensive of which imputes 12 cellular subtypes(20). However, these methods have not yet been extended to the newest EPICv2 array used in the present study. Thus, the inventors derived new models based on overlapping EPICv1 and EPICv2 array CpGs utilizing the previous OA DNA methylation dataset(17) and applied these models to the current dataset. The inventors found two cellular subsets that demonstrated a trend (eosinophils and monocytes, both trended towards an increase in incident OA cases). Importantly, atopic disease, traditionally associated with eosinophilic activation, has recently been strongly associated with knee OA development(23); similarly, monocyte activation is a feature of knee OA(24). However, using imputed cellular composition data alone did not result in models predictive of incident OA.

Using the methylation data, the inventors generated highly accurate models to predict incident OA. The initial screening steps to identify which methylation loci were associated with incident OA yielded 33 CpGs. Although this is a relatively small number, it is similar to the inventors' previous study using peripheral blood methylation patterns to predict future rapid progression in patients with established knee OA in which the inventors identified 13 CpGs as highly predictive of progression(17). Intriguingly, these same 13 CpGs could also be used to develop models with moderate accuracy to predict radiographic progression in the Incident cohort (23 of 33 CpGs were available in the progression cohort), suggesting a conserved epigenetic biomarker potential of these specific locations. Indeed, the present study overcomes the problems of their prior peripheral blood epigenetic modeling, that predicted future rapid progression in established OA patients(16,17). The positive findings in both incident OA and progressive OA cohorts strongly demonstrate epigenetic inflammatory dysregulation occurring throughout the OA disease process.

From a biomarker perspective, the present incident models are superior to previous protein-based models. For example, Kraus and colleagues published in 2024 a study evaluating serum biomarkers in a cohort of 200 women (100 incident radiographic knee OA cases and 100 age- and BMI-matched controls) from the Chingford cohort in the UK. This study differs from the present disclosure in a few ways, most importantly, Kraus and colleagues excluded any participants with a baseline KLG>1, and their definition of incident OA was KLG>1. They performed serum proteomics measurement at years 2 and 6, then developed models for prediction of progression at year 8. Models developed using 11 total serum analytes from a single baseline timepoint demonstrated an AUC of 0.76, while adding demographic, pain, and hip OA information increased this to 0.83(6). Importantly, these AUC values represented modeling on 10-fold cross-validation using an elastic net approach (similar to the development pipeline), but models were not tested on an independent validation set; thus, they represent values similar to the ‘development’ AUCs. A similar study (using serum biomarkers) published by Paz-Gonzalez and colleagues(7) included a smaller cohort of patients (29 incident with 253 controls in the development set and 100 patients in the validation set) and yielded an AUC of 0.83.

Other groups have developed models to predict incident knee OA based on radiographic data. The largest of these, the Knee OsteoArthritis Prediction (KNOAP2020) challenge, brought together seven teams of competitors to develop models based on a test set of 423 knees from the Prevention of Knee Osteoarthritis in Overweight Females study, and included MRI/X-ray image and clinical data(8); the majority of these models were trained on data from the OAI. The highest-performing model demonstrated an AUC of 0.64, combining X-ray and clinical data, although the accuracy of these models was weak (0.55). Another MRI-based modeling approach that defined incident OA like the inventors do (KLG≥2), was published by Joseph et al in 2022. This study had 183 incident OA cases and 861 controls, and their models included pain, physical activity, demographic information, and MRI measures, yielding an AUC of 0.79(9). Thus, the machine learning model and the results as applied to patient samples were clearly superior to prior methodologies.

The 33 CpG sites identified in the present study as predictive of incident OA are, for the most part, located in intergenic regions. However, 9 CpGs are proximate to coding regions. The most strongly predictive CpG site in the models was located in a CpG island near L3MBTL1, an epigenetic effector encoding a polycomb group gene that participates in chromatin modification and is a tumor suppressor gene, not previously linked to OA(25). Urate oxidase (UOX) is another gene with a significant proximate CpG site; this is the pseudogene (mutated and nonfunctional in humans and great apes) that converts uric acid to allantoin(26). SEPTIN9, also in the models, is a cytoskeletal GTPase involved in cytokinesis, vesicle trafficking, DNA repair, cell migration, and apoptosis; and is not previously associated with OA(27). TOLLIP encodes a toll-interacting inhibitory protein that is a component of the IL1/toll-like receptor signaling pathway. Although not previously associated with OA, TOLLIP plays a key role in modulating the innate immune system and lack of Tollip in mice results in chronic low-grade inflammation, especially gut mucosal inflammation and lipopolysaccharide (LPS) responses, atherosclerosis, etc.(28). GLT1D1 is an enzyme that transfers glycosyl groups to proteins (identified in lymphoma), but also has some immunoregulatory function via glycosylation of PD-L1(31), and variants in GLT1D1 have been associated with pediatric-onset lupus(32). Ankyrin 3 (ANK3) is important in sodium ion channels and neuronal excitability, mainly associated with bipolar disorder and intellectual disability(33), although upregulated ANK expression has been shown to promote OA via chondrocyte extracellular pyrophosphate(34). RGS9BP is a regulator of G protein signaling and has been shown to be downregulated in human OA cartilage(35). IFI27L1 is an interferon alpha-inducible protein not previously linked to OA but that has been associated with autoimmune arthritis(36). Finally, PYGM encodes a glycogen phosphorylase within muscle tissue and is a cause of McArdle disease (a glycogen storage myopathy), and is not previously associated with OA(37)/

It was found that peripheral blood DNA methylation data demonstrates superior predictive capability for future incident radiographic OA based on a single baseline blood sample compared to previous biochemical and radiographic (and combinations thereof) biomarker models. Furthermore, the approach included patients of both sexes, unlike previous biochemical biomarker studies that focused exclusively on women. This predictive capability was stable throughout the tested incident timeline (24 m to 96 m). Imputed cell counts and baseline demographic information did not alter model performance, likely due to the careful matching of cases and controls. A relatively small number of CpG sites (n=33) were necessary for generating predictions, making high-throughput clinically applicable testing applicable. Furthermore, these CpG sites also demonstrated the capacity for predicting progression in established OA patients, further widening applicability.

In summary, these studies demonstrate that peripheral blood DNA methylation patterns can be used as biomarkers to predict the future onset of incident knee OA. Taken in combination with the previous analysis of similar patterns as predictive of future progression in established knee OA patients, these studies strongly show that peripheral blood immune cell epigenetic changes may accompany OA throughout the disease course. Herein, the inventors identified a relatively small subset of CpG sites that is strongly correlated with radiographic OA onset and are useful for predicting OA progression in established patients. The AUC in the present study for incident prediction is the highest yet published. These results show that a single-timepoint peripheral blood-based biomarker can be used to define a preclinical OA state for treatment and to improve the outcome of knee OA patients.

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

The functions of the various elements, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “of” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

### REFERENCES

1. Centers for Disease Control and Prevention (CDC). Prevalence of doctor-diagnosed arthritis and arthritis-attributable activity limitation—United States, 2007-2009. MMWR Morb Mortal Wkly Rep 2010; 59:1261-1265.
2. GBD 2021 Osteoarthritis Collaborators. Global, regional, and national burden of osteoarthritis, 1990-2020 and projections to 2050: a systematic analysis for the Global Burden of Disease Study 2021. Lancet Rheumatol 2023; 5:e508-e522.
3. Losina E, Thornhill T S, Rome B N, Wright J, Katz J N. The dramatic increase in total knee replacement utilization rates in the United States cannot be fully explained by growth in population size and the obesity epidemic. J Bone Joint Surg Am 2012; 94:201-207.
4. Fukui N, Yamane S, Ishida S, Tanaka K, Masuda R, Tanaka N, et al. Relationship between radiographic changes and symptoms or physical examination findings in subjects with symptomatic medial knee osteoarthritis: a three-year prospective study. BMC Musculoskelet Disord 2010; 11:269.
5. Gerlag D M, Safy M, Maijer K I, Tang M W, Tas S W, Starmans-Kool M J F, et al. Effects of B-cell directed therapy on the preclinical stage of rheumatoid arthritis: the PRAIRI study. Ann Rheum Dis 2019; 78:179-185.
6. Kraus V B, Sun S, Reed A, Soderblom E J, Moseley M A, Zhou K, et al. An osteoarthritis pathophysiological continuum revealed by molecular biomarkers. Sci Adv 2024; 10:eadj6814.
7. Paz-González R, Balboa-Barreiro V, Lourido L, Calamia V, Fernandez-Puente P, Oreiro N, et al. Prognostic model to predict the incidence of radiographic knee osteoarthritis. Ann Rheum Dis 2024; 83:661-668.
8. Hirvasniemi J, Runhaar J, Heijden R A van der, Zokaeinikoo M, Yang M, Li X, et al. The KNee OsteoArthritis Prediction (KNOAP2020) challenge: An image analysis challenge to predict incident symptomatic radiographic knee osteoarthritis from MRI and X-ray images. Osteoarthritis Cartilage 2023; 31:115-125.
9. Joseph G B, McCulloch C E, Nevitt M C, Link T M, Sohn J H. Machine learning to predict incident radiographic knee osteoarthritis over 8 Years using combined M R imaging features, demographics, and clinical factors: data from the Osteoarthritis Initiative. Osteoarthritis Cartilage 2022; 30:270-279.
10. Handy D E, Castro R, Loscalzo J. Epigenetic modifications. Circulation 2011; 123:2145-2156.
11. Jeffries M A, Donica M, Baker L W, Stevenson M E, Annan A C, Beth Humphrey M, et al. Genome-Wide DNA Methylation Study Identifies Significant Epigenomic Changes in Osteoarthritic Subchondral Bone and Similarity to Overlying Cartilage. Arthritis Rheumatol 2016; 68:1403-1414.
12. Jeffries M A, Donica M, Baker L W, Stevenson M E, Annan A C, Humphrey M B, et al. Genome-wide DNA methylation study identifies significant epigenomic changes in osteoarthritic cartilage. Arthritis Rheumatol 2014; 66:2804-2815.
13. Rushton M D, Reynard L N, Barter M J, Refaie R, Rankin K S, Young D A, et al. Characterization of the cartilage DNA methylome in knee and hip osteoarthritis. Arthritis Rheumatol 2014; 66:2450-2460.
14. Hollander W den, Ramos Y F M, Bos S D, Bomer N, Breggen R van der, Lakenberg N, et al. Knee and hip articular cartilage have distinct epigenomic landscapes: implications for future cartilage regeneration approaches. Ann Rheum Dis 2014; 73:2208-2212.
15. Fernández-Tajes J, Soto-Hermida A, Vázquez-Mosquera M E, Cortes-Pereira E, Mosquera A, Fernández-Moreno M, et al. Genome-wide DNA methylation analysis of articular chondrocytes reveals a cluster of osteoarthritic patients. Ann Rheum Dis 2014; 73:668-677.
16. M Dunn C, Nevitt M C, Lynch J A, Jeffries M A. A pilot study of peripheral blood DNA methylation models as predictors of knee osteoarthritis radiographic progression: data from the Osteoarthritis Initiative (OAI). Sci Rep 2019; 9:16880.
17. Dunn C M, Sturdy C, Velasco C, Schlupp L, Prinz E, Izda V, et al. Peripheral Blood DNA Methylation-Based Machine Learning Models for Prediction of Knee Osteoarthritis Progression: Biologic Specimens and Data From the Osteoarthritis Initiative and Johnston County Osteoarthritis Project. Arthritis Rheumatol 2023; 75:28-40.
18. Wirth W, Duryea J, Hellio Le Graverand M-P, John M R, Nevitt M, Buck R J, et al. Direct comparison of fixed flexion, radiography and MRI in knee osteoarthritis: responsiveness data from the Osteoarthritis Initiative. Osteoarthritis Cartilage 2013; 21:117-125.
19. Akulenko R, Merl M, Helms V. BEclear: Batch effect detection and adjustment in DNA methylation data. PLoS One 2016; 11:e0159921.
20. Salas L A, Zhang Z, Koestler D C, Butler R A, Hansen H M, Molinaro A M, et al. Enhanced cell deconvolution of peripheral blood using DNA methylation for high-resolution immune profiling. Nat Commun 2022; 13:761.
21. Houseman E A, Accomando W P, Koestler D C, Christensen B C, Marsit C J, Nelson H H, et al. DNA methylation arrays as surrogate measures of cell mixture distribution. BMC Bioinformatics 2012; 13:86.
22. Aryee M J, Jaffe A E, Corrada-Bravo H, Ladd-Acosta C, Feinberg A P, Hansen K D, et al. Minfi: a flexible and comprehensive Bioconductor package for the analysis of Infinium DNA methylation microarrays. Bioinformatics 2014; 30:1363-1369.
23. Baker M C, Sheth K, Lu R, Lu D, Kaeppler E P von, Bhat A, et al. Increased risk of osteoarthritis in patients with atopic disease. Ann Rheum Dis 2023:ard-2022-223640.
24. Loukov D, Karampatos S, Maly M R, Bowdish D M E. Monocyte activation is elevated in women with knee-osteoarthritis and associated with inflammation, BMI and pain. Osteoarthritis Cartilage 2018; 26:255-263.
25. Gurvich N, Perna F, Farina A, Voza F, Menendez S, Hurwitz J, et al. L3MBTL1 polycomb protein, a candidate tumor suppressor in del(20q12) myeloid disorders, is essential for genome stability. Proc Natl Acad Sci USA 2010; 107:22552-22557.
26. Oda M, Satta Y, Takenaka O, Takahata N. Loss of urate oxidase activity in hominoids and its evolutionary implications. Mol Biol Evol 2002; 19:640-653.
27. Sun J, Zheng M-Y, Li Y-W, Zhang S-W. Structure and function of Septin 9 and its role in human malignant tumors. World J Gastrointest Oncol 2020; 12:619-631.
28. Kowalski E J A, Li L. Toll-interacting protein in resolving and non-resolving inflammation. Front Immunol 2017; 8:511.
29. Huang Z Y, Stabler T, Pei F X, Kraus V B. Both systemic and local lipopolysaccharide (LPS) burden are associated with knee OA severity and inflammation. Osteoarthritis Cartilage 2016; 24:1769-1775.
30. Griffin T M, Scanzello C R. Innate inflammation and synovial macrophages in osteoarthritis pathophysiology. Clin Exp Rheumatol 2019; 37 Suppl 120:57-63.
31. Liu X, Zhang Y, Han Y, Lu W, Yang J, Tian J, et al. Overexpression of GLT1D1 induces immunosuppression through glycosylation of PD-L1 and predicts poor prognosis in B-cell lymphoma. Mol Oncol 2020; 14:1028-1044.
32. Joo Y B, Lim J, Tsao B P, Nath S K, Kim K, Bae S-C. Genetic variants in systemic lupus erythematosus susceptibility loci, XKR6 and GLT1D1 are associated with childhood-onset SLE in a Korean cohort. Sci Rep 2018; 8:9962.
33. Hughes T, Sønderby I E, Polushina T, Hansson L, Holmgren A, Athanasiu L, et al. Elevated expression of a minor isoform of ANK3 is a risk factor for bipolar disorder. Transl Psychiatry 2018; 8:210.
34. Johnson K, Terkeltaub R. Upregulated ank expression in osteoarthritis can promote both chondrocyte MMP-13 expression and calcification via chondrocyte extracellular PPi excess. Osteoarthritis Cartilage 2004; 12:321-335.
35. Murab S, Chameettachal S, Bhattacharjee M, Das S, Kaplan D L, Ghosh S. Matrix-embedded cytokines to simulate osteoarthritis-like cartilage microenvironments. Tissue Eng Part A 2013; 19:1733-1753.
36. Tyagi N, Mehla K, Gupta D. Deciphering novel common gene signatures for rheumatoid arthritis and systemic lupus erythematosus by integrative analysis of transcriptomic profiles. PLoS One 2023; 18:e0281637.
37. Núñez-Manchón J, Ballester-Lopez A, Koehorst E, Linares-Pardo I, Coenen D, Ara I, et al. Manifesting heterozygotes in McArdle disease: a myth or a reality-role of statins. J Inherit Metab Dis 2018; 41:1027-1035.

What is claimed is:

1. A method for determining whether a subject is at increased risk of developing future knee osteoarthritis within 12 to 96 months, the method comprising the steps of:
(a) obtaining, or having obtained, a blood sample;
(b) measuring in the blood sample a DNA methylation level at one or more DNA methylation sites;
(c) comparing the DNA methylation level(s) measured in step (b) to level(s) of DNA methylation in a control blood sample from an individual without osteoarthritis;
(d) using a machine learning algorithm to generate an osteoarthritis risk score indicative of a likelihood of developing knee osteoarthritis based on the comparison of methylation levels; and
(e) based on the osteoarthritis risk score identifying the subject as being at increased risk for future knee osteoarthritis within 12 to 96 months.

2. The method of claim 1, wherein the blood sample comprises peripheral blood leukocytes.

3. The method of claim 1, further comprising at least one of:
preprocessing a methylation array data by: excluding from analysis CpG probes located on sex chromosomes, probes with known single nucleotide polymorphisms (SNPs) with minor allele frequency of ≥5%, and probes not detected in all samples; or
wherein the individual without osteoarthritis: (i) is matched to the subject by at least one of: age category, sex, BMI category, ethnicity, or baseline KL radiographic grade; and (ii) does not have osteoarthritis as determined by both radiographic and symptomatic examination.

4. The method of claim 1, wherein the machine learning algorithm is selected from a generalized logistic model, a parsimonious model, or combinations thereof.

5. The method of claim 1, wherein a control blood level of the DNA methylation is a predetermined standard from an individual that will not develop osteoarthritis within the subsequent 96 months as determined by radiographic and symptomatic examination.

6. The method of claim 1, wherein the DNA methylation is determined with an Illumina 450k, an EPIC v1, an EPIC v2 array, or CpG sites associates with at least one of: cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; or cg07592337.

7. The method of claim 1, wherein the DNA methylation analyzed further comprises in order of importance 1, 2, 3, 4, 5, 6, 7, 8, 9, 10, 11, 12, 13, 14, 15, 16, 17, 18, 19, 20, 25, 30, or 33 of the following biomarkers cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; and cg07592337.

8. The method of claim 1, wherein the subject is human.

9. The method of claim 1, wherein the subject has a Kellgren-Lawrence (KL) score of 0-1 in at least one knee.

10. The method of claim 1, further comprising the step of treating the osteoarthritis with at least one of acetaminophen, steroids, hyaluronic acid, nonsteroidal anti-inflammatory drugs (NSAIDs), or duloxetine, once the subject has a KL score of ≥2.

11. The method of claim 1, wherein the DNA methylation is a CpG island associated with 1, 2, 3, 4, 5, 6, 7, 8, or 9 markers selected from L3MBTL Histone Methyl-Lysine Binding Protein 1 (L3MBTL1), Urate oxidase (UOX), SEPTIN9, Toll-Interacting Protein (TOLLIP), Glycosyltransferase 1 Domain-Containing Protein 1 (GLT1D1), Ankyrin 3 (ANK3), Regulator Of G Protein Signaling 9 Binding Protein (RGS9BP), Interferon Alpha Inducible Protein 27 Like 1 (IFI27L1), and Glycogen Phosphorylase, Muscle Form (PYGM).

12. A method for determining whether to treat a subject at increased risk of developing future knee osteoarthritis within 12 to 96 months, the method comprising the steps of:
(a) obtaining, or having obtained, DNA methylation level(s) at one or more CpG sites from a blood sample of the subject;
(b) comparing the DNA methylation level(s) measured in step (a) to DNA methylation level(s) at one or more CpG sites in a control blood sample from an individual without osteoarthritis;
(c) calculating using a machine learning algorithm to generate an osteoarthritis risk score indicative of risk of future knee osteoarthritis based on the comparison of methylation levels;
(d) based on the osteoarthritis risk score identifying the subject as being at increased risk for future knee osteoarthritis within 12 to 96 months with the osteoarthritis risk score generated in step (c) that is indicative that the subject is at risk of knee osteoarthritis within 12 to 96 months; and
(e) if the subject has osteoarthritis risk score for increased risk and a Kellgren-Lawrence (KL) score of ≥2, then administering to the subject with at least one of acetaminophen, steroids, hyaluronic acid, nonsteroidal anti-inflammatory drugs (NSAIDs), or duloxetine in an amount effective to reduce knee osteoarthritis.

13. The method of claim 12, wherein the subject is at increased risk of knee osteoarthritis and has a Kellgren-Lawrence (KL) score of 0-1 in at least one knee.

14. The method of claim 12, wherein the blood sample obtained from the subject has been prepared in the presence of heparin.

15. The method of claim 12, wherein the machine learning algorithm is selected from a generalized logistic model, a parsimonious model, or combinations thereof.

16. The method of claim 12, wherein a control blood level of the DNA methylation is a predetermined standard from an individual that does not have osteoarthritis as determined by both radiographic and symptomatic examination.

17. The method of claim 12, wherein the DNA methylation is determined with an Illumina 450k, an EPIC v1, an EPIC v2 array, or CpG sites associates with at least one of: cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; or cg07592337.

18. The method of claim 12, wherein the DNA methylation analyzed further comprises in order of importance 1, 2, 3, 4, 5, 6, 7, 8, 9, 10, 11, 12, 13, 14, 15, 16, 17, 18, 19, 20, 25, 30, or 33 of the following biomarkers cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; and cg07592337.

19. The method of claim 12, wherein the DNA methylation level(s) in the control blood sample is a level of DNA methylation in a similarly processed blood sample from an individual without osteoarthritis or a mean level of several individuals without osteoarthritis.

20. The method of claim 12, wherein the individual without osteoarthritis (i) is matched to the subject by at least one of: age category, sex, BMI category, ethnicity, or baseline KL radiographic grade; and (ii) does not have osteoarthritis as determined by both radiographic and symptomatic examination.

21. The method of claim 12, wherein the DNA methylation is a CpG island associated with 1, 2, 3, 4, 5, 6, 7, 8, or 9 markers selected from L3MBTL Histone Methyl-Lysine Binding Protein 1 (L3MBTL1), Urate oxidase (UOX), SEPTIN9, Toll-Interacting Protein (TOLLIP), Glycosyltransferase 1 Domain-Containing Protein 1 (GLT1D1), Ankyrin 3 (ANK3), Regulator Of G Protein Signaling 9 Binding Protein (RGS9BP), Interferon Alpha Inducible Protein 27 Like 1 (IFI27L1), and Glycogen Phosphorylase, Muscle Form (PYGM).

22. A method for determining whether a subject is at increased risk of developing future knee osteoarthritis, wherein the subject does not have osteoarthritis but is predicted to have knee osteoarthritis in 0 to 96 months, the method comprising the steps of:
(a) comparing DNA methylation level(s) measured at one or more CpG sites from a blood sample of the subject to DNA methylation level(s) at one or more CpG sites in a control blood sample from an individual without osteoarthritis;
(b) calculating using a machine learning algorithm to generate an osteoarthritis risk score, wherein the machine learning algorithm is selected from a generalized logistic model, a parsimonious model, or combinations thereof; and
(c) based on the osteoarthritis risk score identifying the subject as being at increased risk for developing future osteoarthritis of the knee within 0 to 96 months when the osteoarthritis risk score is equal to, or greater than, 73% accuracy,
wherein the DNA methylation is determined for at least 1, 2, 3, 4, 5, 6, 7, 8, 9, or 10 markers selected from: cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; or cg07592337.

23. The method of claim 22, wherein the DNA methylation analyzed further comprises in order of importance 1, 2, 3, 4, 5, 6, 7, 8, 9, 10, 11, 12, 13, 14, 15, 16, 17, 18, 19, 20, 25, 30, or 33 of the following biomarkers cg06908806; cg13731777; cg17976021; cg01095449; cg14471784; cg06601130; cg19922101; cg14938831; cg27387124; cg05102645; cg22424444; cg08111925; cg12257696; cg16478864; cg07328664; cg26395513; cg02918525; cg18016034; cg12597363; cg15480200; cg05946856; cg27413643; cg06736189; cg27560391; cg19093112; cg21743307; cg07553358; cg13532410; cg13606440; cg05139637; cg10972590; cg22668906; and cg07592337.

24. The method of claim 22, wherein the methylation of the 1, 2, 3, 4, 5, 6, 7, 8, 9, or 10 markers is selected, in the following order: cg06908806; cg13731777; cg17976021; cg01095449; cg14471784; cg06601130; cg19922101; cg14938831; cg27387124; and cg05102645.

25. The method of claim 22, wherein the subject has not been diagnosed with knee osteoarthritis and/or has a Kellgren-Lawrence (KL) score of 0-1.

26. The method of claim 22, wherein the subject is human.

27. The method of claim 22, wherein an osteoarthritis treatment, once the subject has a KL score of ≥2, is at least one of acetaminophen, steroids, hyaluronic acid, nonsteroidal anti-inflammatory drugs (NSAIDs), or duloxetine.

28. The method of claim 22, wherein an osteoarthritis disease risk is identified 6, 12, 24, 36, 48, 60, 72, or 96 months prior to a Kellgren-Lawrence (KL) disease severity score ≥2, or ≥3.

29. A computer-implemented system for determining whether a subject is at increased risk of developing future knee osteoarthritis within 12 to 96 months comprising:
a hardware processor and a memory coupled to the hardware processor, wherein the memory comprises a set of instructions in the form of a processing subsystem, configured to be executed by the hardware processor, comprises:
obtaining, or having obtained, a blood sample;
measuring in the blood sample a DNA methylation level at one or more DNA methylation sites;
comparing the DNA methylation level(s) measured in step (b) to level(s) of DNA methylation in a control blood sample from an individual without osteoarthritis;
using a machine learning algorithm to generate an osteoarthritis risk score indicative of a likelihood of developing knee osteoarthritis based on the comparison of methylation levels; and
based on the osteoarthritis risk score identifying the subject as being at increased risk for future knee osteoarthritis within 12 to 96 months.

30. The computer-implemented system of claim 29, wherein the knee osteoarthritis risk score model is generated by obtaining a complete dataset of knee osteoarthritis samples and expression data, selecting features using lasso regression (L1 penalization of a generalized logistic regression model) and CpG sites were identified as highly predictive of incident OA cases vs. controls; applying a Monte Carlo cross-validation step in which the data are split into 60 to 80% development and 20 to 40% validation subsets and modeling performed on the development set with a similar 10-fold cross-validated approach; splitting randomly 1:10 the data in the 60 to 80% development step datal applying a 10-fold cross-validated model development of the CpG+/− covariates available from glmnet models, repeating the random split 5, 6, 7, 8, 9, 10, 11, 12, 13, 14, or 15 times; after the nth repeat conducting further feature reduction and model tuning; optimizing the model 28, by testing against unseen data as a validation step.

31. The computer-implemented system of claim 29, further cross-validating by looping random data splitting and model development for a total of 10, 20, 25, 30, 35, 40, 45, 50, or 60 cycles by Monte Carlo cross-validation, and splitting the data again split into random groups and is split into the Monte Carlo cross-validation step, and calculating a mean model performance for CpG sites selected to obtain the CpG sites used to calculate the knee osteoarthritis risk score. Further comprising applying the knee osteoarthritis risk score model to a lockbox set to determine at least one of: area under the receiver operator characteristic curve (AUC), diagnostic odds ratio, accuracy, sensitivity, specificity, or F1 score; and DNA methylation sites.

32. The computer-implemented system of claim 29, further comprising the step of treating the osteoarthritis with at least one of acetaminophen, steroids, hyaluronic acid, nonsteroidal anti-inflammatory drugs (NSAIDs), or duloxetine, once the subject has a KL score of ≥2.

33. A non-transitory computer-readable medium storing a computer program that, when executed by a processor, causes the processor to perform a method for determining whether a subject is at increased risk of developing future knee osteoarthritis within 12 to 96 months, wherein the method comprises:
interfacing with a machine learning model that generate a knee osteoarthritis risk score;
automatically processing, a knee osteoarthritis risk score module by:
obtaining, or having obtained, a blood sample;
measuring in the blood sample a DNA methylation level at one or more DNA methylation sites;
comparing the DNA methylation level(s) measured in step (b) to level(s) of DNA methylation in a control blood sample from an individual without osteoarthritis;
using a machine learning algorithm to generate an osteoarthritis risk score indicative of a likelihood of developing knee osteoarthritis based on the comparison of methylation levels; and
based on the osteoarthritis risk score identifying the subject as being at increased risk for future knee osteoarthritis within 12 to 96 months.

34. The non-transitory computer-readable medium of claim 33, wherein the knee osteoarthritis risk score model is generated by obtaining a complete dataset of knee osteoarthritis samples and expression data, selecting features using lasso regression (L1 penalization of a generalized logistic regression model) and CpG sites were identified as highly predictive of incident OA cases vs. controls; applying a Monte Carlo cross-validation step in which the data are split into 60 to 80% development and 20 to 40% validation subsets and modeling performed on the development set with a similar 10-fold cross-validated approach; splitting randomly 1:10 the data in the 60 to 80% development step datal applying a 10-fold cross-validated model development of the CpG+/− covariates available from glmnet models, repeating the random split 5, 6, 7, 8, 9, 10, 11, 12, 13, 14, or 15 times; after the nth repeat conducting further feature reduction and model tuning; optimizing the model 28, by testing against unseen data as a validation step.

35. The non-transitory computer-readable medium of claim 33, further cross-validating by looping random data splitting and model development for a total of 10, 20, 25, 30, 35, 40, 45, 50, or 60 cycles by Monte Carlo cross-validation, and splitting the data again split into random groups and is split into the Monte Carlo cross-validation step, and calculating a mean model performance for CpG sites selected to obtain the CpG sites used to calculate the knee osteoarthritis risk score. Further comprising applying the knee osteoarthritis risk score model to a lockbox set to determine at least one of: area under the receiver operator characteristic curve (AUC), diagnostic odds ratio, accuracy, sensitivity, specificity, or F1 score; and DNA methylation sites.

36. The non-transitory computer-readable medium of claim 33, further comprising the step of administering to the subject with the osteoarthritis risk score identifying the subject as being at increased risk for future knee osteoarthritis at least one of acetaminophen, steroids, hyaluronic acid, nonsteroidal anti-inflammatory drugs (NSAIDs), or duloxetine.
